(19) United States
(12) Patent Application Publication
(10) Pub. No.: US 2009/0232746 A1
(43) Pub. Date: Sep. 17, 2009

(54) SILICONE POLYMER-SILICA SYSTEM

(76) Inventors: Juan R. Mateu, Oak Ridge, NJ (US); Salvatore J. Barone, Staten Island, NY (US); Irina Staina, Branchburg, NJ (US); Ralph Macchio, Sparta, NJ (US)

Correspondence Address: SCHWEGMAN, LUNDBERG & WOESSNER, P.A., P.O. BOX 2938, MINNEAPOLIS, MN, 55402, US

(21) Appl. No.: 12/275,940

(22) Filed: Nov. 21, 2008

Related U.S. Application Data
(60) Provisional application No. 60/991,556, filed on Nov. 30, 2007.

(51) Int. Cl. A61K 9/12; A61K 8/25; A61Q 15/00

(52) U.S. Cl. 424/47; 424/65

(57) ABSTRACT

A chemical system comprising amodimethicone, dimethicone gum and silica is provided. The chemical system is useful in varying concentrations in antiperspirants of any form, including, but not limited to, solid stick, aerosol spray and liquid roll-on. Embodiments of the invention further include a method for preparing a chemical composition comprising amodimethicone, dimethicone gum and silica

### CROSS-REFERENCE TO RELATED APPLICATION

This application claims the benefit of U.S. Provisional Patent Application Ser. No. 60/991,556 filed Nov. 30, 2007, which is incorporated herein by reference and made a part hereof.

### FIELD OF THE INVENTION

Embodiments of the invention described herein relate to a silicone polymer-silica system, one or more methods for making the silicone polymer-silica system and formulations that include the silicone polymer-silica system.

### BACKGROUND

Antiperspirants have, as a principle function, a requirement to eliminate adverse effects of perspiration. Antiperspirant function has frequently been associated with undesirable side effects.

### SUMMARY

One embodiment of the invention described herein includes a chemical system comprising amodimethicone, dimethicone gum and silica. The chemical system is useful in varying concentrations in antiperspirants of any form. In various embodiments, each component may have a concentration of anywhere from 1 to 99%. In one embodiment, the chemical system further comprises packaging for transporting and storing the chemical system. The chemical system may further comprise water and/or one or more active deodorancy components.

One embodiment of the invention further includes a method for preparing a chemical composition comprising preparing a chemical system comprising amodimethicone, dimethicone gum and silica; and providing the chemical system to a manufacturer of the chemical composition. In one embodiment, the method further includes packaging the chemical system prior to providing the system to the manufacturer. In one embodiment, the method further includes preparing the amodimethicone, dimethicone gum and with ratios for the chemical composition manufactured by the manufacturer.

Embodiments of the invention further comprise an antiperspirant comprising a chemical system comprising amodimethicone; dimethicone gum; and silica. In one embodiment, the antiperspirant is an aerosol spray. In one embodiment, the antiperspirant is a solid stick. In one embodiment, the antiperspirant is a liquid roll-on. In one embodiment, the antiperspirant is a deodorant antiperspirant.

Antiperspirants formulated from embodiments of the chemical system described herein are expected to have increased efficacy and enhanced residual skin feel due to the presence of amodimethicone, improved skin adherence due to the presence of dimethicone gum and an improved overall structure due to the presence of silica. Use of silica further improves the efficacy of solid stick antiperspirants.

### DESCRIPTION

Invention embodiments are described in sufficient detail to enable those skilled in the art to practice the invention. Other embodiments may be utilized and structural, logical, chemical, and other changes may be made without departing from the spirit or scope of the invention discussed herein. The following detailed description is, therefore, not to be taken in a limiting sense, and the scope of the invention discussed herein is defined only by the appended claims.

Embodiments of the invention described herein include a base system for use in antiperspirant compositions, as well as formulations that include the base system, and methods for making the base system.

The base system includes a blend of amodimethicone, dimethicone gum and silica. The concentration of each component in the system is variable and depends upon the application of the base system. A specific concentration may be used in order to maximize efficacy, while minimizing cost and stability issues.

Amodimethicone or aminopropyl dimethicone is an amine-containing silicone polymer useful for multi-product applications. See, for example, U.S. Pat. No. 6,649,151 to Applicant, which issued on Nov. 18, 2003, incorporated herein by reference in its entirety. In the acidic antiperspirant environment, the amine groups in amodimethicone are positively charged. The positive charge gives the dimethicone additional properties not usually associated with silicone polymers. These properties are useful across all the antiperspirant product forms. For example, amodimethicone is a potent water absorber and further acts as an emulsifier to stabilize oil (silicone) and water mixtures. The ability to act as an emulsifier provides a pathway for water to penetrate the oil phase and decrease the “water-proofing” affect associated with silicone and water insoluble oils. As a result, efficacy is increased, because distribution of the antiperspirant active is improved, thus making it more available to diffuse into and block the sweat ducts. The positive charge also makes the silicone more substantive to skin for enhanced residual skin feel.

Dimethicone gum or dimethicone is a high molecular weight silicone polymer. Dimethicone gum is a film former useful in all forms of antiperspirants which promotes better adherence of antiperspirant actives to the skin and reduces rub off. In sprays, the high molecular weight of the polymer alters the physical properties of aerosolized droplets resulting in improved adherence to the skin, making them less likely to bounce off the skin. As a result, more of the product sticks to the skin and does not bounce off. An equal amount of the product containing the dimethicone gum dispensed from an aerosol container will deposit more antiperspirant active on the skin than a product not containing the dimethicone gum. Generally, more antiperspirant active means better wetness control.

Silica (SiO₂) is an inorganic oxide of silicon. Silica promotes better structures for complex mixtures. Silica is useful across all antiperspirant product forms in the moisture absorbing complex as a binder to help hold the components together. In this way, the complex maintains its uniform properties upon storage and in the final product. Silica further modifies and improves the wax matrix which provides the structure of an antiperspirant stick. The improved matrix allows additional moisture absorbing complex to be added to the stick, thus improving efficacy of the stick.

The above components, namely amodimethicone, dimethicone gum and silica, may be combined in any suitable concentration depending on the particular application. In one embodiment, the amodimethicone is present in an amount ranging from 1 to 99% by weight, including any specific amount, such as 1.1, 1.2, 1.3, 1.4% by weight, and so forth, up to 99.0% by weight. In one embodiment, dimethicone gum is present in an amount ranging from 1 to 99% by weight, including any specific amount, such as 1.1, 1.2, 1.3, 1.4% by weight, and so forth, up to 99.0% by weight. In one embodiment, silica is present in an amount ranging from 1 to 99% by weight, including any specific amount, such as 1.1, 1.2, 1.3, 1.4% by weight, and so forth, up to 99.0% by weight.

Additional components useful in antiperspirants may also be combined with the above components as is known in the art. Such additional components include, but are not limited to, components useful for providing uniform application and resistance to staining. It is desirable that antiperspirants also be gentle to skin and minimize loss of moisture from skin to which antiperspirant is applied. In one embodiment, the additional components may include skin conditioners. In one embodiment, the antiperspirant is an antiperspirant deodorant which contains active components known in the art to contribute to deodorancy. In one embodiment, fragrances known in the art are used. In one embodiment, water, such as de-ionized water, is an optional component in the systems described herein.

The various systems described herein are useful in a wide range of antiperspirant products. Examples of the physical states of this product include solids, semi-solids, and liquids. It is also possible to use this product by spraying. As such, the various systems described herein are useful in aerosol sprays, solid sticks and liquid roll-ons. The various systems described herein may also be useful in other products, including, but not limited to, repellents (e.g., insect repellents), sunscreens and emollients.

Although specific embodiments have been illustrated and described herein, it will be appreciated by those of ordinary skill in the art that any procedure that is calculated to achieve the same purpose may be substituted for the specific embodiments shown. This application is intended to cover any adaptations or variations of the present subject matter. Therefore, it is manifestly intended that embodiments of this invention be limited only by the claims and the equivalents thereof.

What is claimed is:

1. A chemical system comprising amodimethicone, dimethicone gum and silica.

2. The chemical system of claim 1, further comprising packaging for transporting and storing the chemical system.

3. The chemical system of claim 1, wherein the amodimethicone has a concentration of 1 to 99% by weight.

4. The chemical system of claim 1, wherein the dimethicone gum has a concentration of 1 to 99% by weight.

5. The chemical system of claim 1, wherein the silica has a concentration of 1 to 99% by weight.

6. The chemical system of claim 1, further comprising water.

7. The chemical system of claim 1, further comprising one or more active deodorancy components.

8. A method for preparing a chemical composition comprising:
preparing a chemical system comprising amodimethicone, dimethicone gum and silica; and
providing the chemical system to a manufacturer of the chemical composition.

9. The method of claim 8, further comprising packaging the chemical system prior to providing the system to the manufacturer.

10. The method of claim 9, wherein the amodimethicone, dimethicone gum and silica are prepared with ratios for the chemical composition manufactured by the manufacturer.

11. The method of claim 8, wherein the chemical system further comprises water.

12. The method of claim 8, wherein the chemical system further comprises one or more active deodorancy components.

13. An antiperspirant comprising:
a chemical system, comprising:
amodimethicone;
dimethicone gum; and
silica.

14. The antiperspirant of claim 13 wherein the antiperspirant is an aerosol spray.

15. The antiperspirant of claim 13 wherein the antiperspirant is a solid stick.

16. The antiperspirant of claim 13 wherein the antiperspirant is a liquid roll-on.

17. The antiperspirant of claim 13, wherein the antiperspirant is a deodorant antiperspirant.
